(19) United States
(12) Patent Application Publication
(10) Pub. No.: US 2008/0168707 A1
(43) Pub. Date: Jul. 17, 2008

(54) Additive for hydrocarbon fuel and related processes consisting of compounds of adenosine phosphates

(75) Inventors: C. Edward Baxter, League City, TX (US); Robert Ehrlich, Follett, TX (US)

Correspondence Address: BRACEWELL & GIULIANI LLP, P.O. BOX 61389, HOUSTON, TX, 77208-1389, US

(73) Assignee: EnviroFuels LLC, Houston, TX (US)

(21) Appl. No.: 11/804,070

(22) Filed: May 17, 2007

(30) Foreign Application Priority Data
Jan. 17, 2007 (US) PCT/US07/01152

(51) Int. Cl. C10L 1/222; C07H 19/04; C10L 10/00

(52) U.S. Cl. 44/336; 44/603; 536/26.7

(57) ABSTRACT

The present invention relates to the field of fuel additives, in particular, to an additive for hydrocarbon fuels to enhance efficiency and/or reduce pollution. The fuel additive of the invention includes an adenosine phosphate. The use of the adenosine phosphate results in enhanced combustion when combined with hydrocarbon fuel and combusted.

### BACKGROUND OF THE INVENTION

Many hydrocarbon fuels have been used, each with their own advantages and drawbacks. Examples of such fuels include gasoline, natural gas, diesel, kerosene, jet fuel, LPG, heavy distillates, bunker fuel, ethanol, coal, other solid hydrocarbon fuels and the like. Chemical compounds have been used as fuel additives over the past century to improve various parameters, such as octane number, of various fuels. The use, and subsequent banning, of lead in gasoline has been known for a long time. Tetraethyl lead showed a positive effect on octane and a profoundly negative effect on the environment.

In addition to tetraethyl lead, several elements are known to have combustion catalyst characteristics in gasoline or other hydrocarbon fuels. Examples, in addition to lead, are manganese, iron, copper, cerium, calcium and barium. Each of these elements has advantages and disadvantages in particular applications. Drawbacks of certain iron compounds include limited solubility in gasoline, toxicity, and expense as an additive. Interaction with sulfur and creation of sulfide precipitate may also occur, which is undesirable.

Another commonly-used additive in gasoline is MTBE. While this compound boosts octane levels significantly, the compound is thought to be carcinogenic. Also, it mixes easily with water which is hazardous should there be a leak. Gasoline containing MTBE leaking from an underground tank at a gas station could potentially leach into groundwater and contaminate wells. As a result of the believed negative potential of MTBE on the environment, ethanol is also being evaluated as a gasoline additive to boost octane.

In addition to the industry goal of improved combustion efficiency, smoke emissions reduction is also a concern, particularly for diesel fuel applications. The industry has not made substantial progress on development of a fuel additive for reducing smoke and particulate emissions. These and other issues are identified in co-pending U.S. application Ser. Nos. 10/875,765 and 11/273,982, both of which are incorporated herein by reference.

Finally, adjustment of combustion parameters is made to attempt to maximize function to reduce CO and NOx. In spite of these and combinations of these attempts to minimize pollutants, fuel combustion continues to be a focus of interest to improve fuel efficiency and reduce pollutants.

Solid hydrocarbon fuels, such as coal, have long been used to fuel burners, furnaces and open flames. Coal, particularly lignite and other low BTU coals, does not burn very efficiently and generates considerable smoke, NOx, SO₂, particulate matter and other undesirable emissions including CO. Chemical compounds have been used as combustion improvers to enhance combustion efficiency, of solid fuels. However, many of these additives contain heavy metallic elements such as manganese, zinc, iron, copper, cerium, calcium and barium. On burning, some of these elements can produce heavy metal solid residues, which are highly undesirable.

A fuel additive that includes a combustion catalyst to reduce smoke and particulate emissions from coal fired burners, furnaces and other direct-fired applications would be advantageous. A fuel additive that increases efficiency and/or decreases pollutants for coal and other solid fuels including low quality coal such as lignite, used in these applications would be particularly advantageous. It would also be advantageous to reduce smoke, particulate and nitrogen-containing emissions from fuel applications. In addition to reduction of NOₓ, reduction or elimination of other toxic pollutants, such as S0₂, is highly desirable. An additive that does not result in the formation of heavy metal precipitates and residues during the combustion process would be desirable.

### SUMMARY OF THE INVENTION

The present invention includes a fuel additive and a method of using the fuel additive in relation to hydrocarbon fuel.

The fuel additive of the invention includes an adenosine phosphate. A particularly preferred adenosine phosphate is adenosine triphosphate (ATP). ATP can be used alone or in combination with any of its forms or homologs. Cationic salts of the adenosine phosphates are also included within the scope of the invention. Typically, such salts are anhydrous salts of ATP as ATP is highly soluble in water. Preferred cations are the alkali metals or Group IA elements. Ammonium cations are also a preferred class of cations. When ammonium cations are present, there is at most a negligible amount of free ammonia such that the resulting combination of fuel additive and fuel are virtually ammonia free.

While adenosine phosphate can advantageously be added directly with the fuel, another preferred embodiment includes creating an adenosine phosphate solution. If the adenosine compounds are soluble in hydrocarbons, such as liquid hydrocarbon fuels, the adenosine phosphate solution includes dissolving the adenosine phosphate directly into an amount of the liquid hydrocarbon fuel or into a hydrocarbon carrier fluid. For example, adding adenosine phosphate into the amount of the liquid hydrocarbon fuel creates a concentrate suitable for storage or transportation. This adenosine phosphate solution can then be added to a greater quantity of liquid hydrocarbon fuel to achieve the desired concentration of adenosine phosphate in fuel. Alternately, adenosine phosphate added to the hydrocarbon carrier fluid to create the adenosine phosphate solution can then be added to the liquid hydrocarbon fuel. The hydrocarbon carrier fluid is soluble in the liquid hydrocarbon fuel. The adenosine phosphate solution created in one of these manners is referred to as hydrocarbon adenosine phosphate solution.

In an alternate embodiment, adenosine phosphate salts that are not completely soluble in hydrocarbon can be dissolved or at least partially dispersed in water or other appropriate carrier fluid to create the adenosine phosphate solution. ATP, for example, is water soluble. In a preferred embodiment, ATP in a neutral solution is ionized and is thought to exists mostly as ATP⁴⁻. The water acts as carrier fluid. Other preferred aqueous carrier fluids include alcohols. Water and alcohol are among the carrier fluids that act as solvents that are referred to as volatile components in that they can be removed from the adenosine phosphate solution through the application of heat. The adenosine phosphate solution created in one of these manners is referred to as aqueous adenosine phosphate solutions.

In another embodiment, when the hydrocarbon fuel is solid hydrocarbon fuel, the adenosine phosphate solution can be added to the hydrocarbon fuel by traditional methods such as spraying or in a slurry.

### DETAILED DESCRIPTION OF THE INVENTION

In a preferred embodiment, the adenosine phosphate solution is added or mixed with a dispersion fluid to create a treated adenosine phosphate solution. The dispersion fluid is a fluid that is operable to maintain the adenosine phosphate within the dispersion fluid in at least a partially dispersed state and that is at least partially miscible, or capable of being maintained in solution (jointly referred to as “miscible”), in the hydrocarbon fuel. A stable emulsion is also considered to be maintained in solution. In certain circumstances, the dispersion fluid is a quantity of a target fluid, that is, a fluid that contains the desired fuel. In a preferred embodiment, the carrier fluid is largely removed from the treated adenosine phosphate solution through thermal means to create the fuel additive. For example, when water is used as carrier fluid, thermal removal is preferred. The fuel additive is operable to enhance combustion when placed into contact with fuel, particularly in an internal combustion engine. Enhanced combustion means that fuel efficiency is increased when compared to fuel without the fuel additive, or that pollutant output in an exhaust gas from the combustion is decreased or a combination of these effects. Typical pollutants can include NOx, particulate matter, carbon monoxide and other recognized pollutants resulting from the combustion of hydrocarbon fuel. It is noted that different geographical areas focus on minimizing a particular pollutant depending on air characteristics. Reduction of a target pollutant or a combination of pollutants, such as NOx and CO is highly advantageous. Alternately, increased fuel efficiency results in a total lower volume of pollutants, as well as economic advantage.

When the fuel additive is prepared using ammonium compounds, ammonium compounds are defined as those compounds containing NRₓ groups, where R can be alkyl, aryl or hydrogen or combinations thereof. A preferred embodiment includes R as hydrogen.

The adenosine phosphate solution can be used in either hydrophilic or hydrophobic environments. In the case of a hydrophobic environment, the carrier fluid is selected to allow for proper dispersion. In one preferred embodiment, a commercially available carrier fluid is used. In another embodiment, a combination of carrier fluids is used. A dispersant agent used in conjunction with the carrier fluids to create the fuel additive is also encompassed in a preferred embodiment. For liquid hydrocarbon fuel applications, at least one carrier fluid can preferably be a fluid with a least some hydrophilic character that is miscible with the fuel to act as compatibilizing agent in conjunction with dispersant agent.

The fuel additive of the invention is useful to enhance combustion such that more complete combustion is achieved with increased combustion to CO₂ and H₂O as compared to the combustion of the fuel without the fuel additive. The outcome is the reduction of products of partial combustion as well as NOₓ, thereby increasing fuel efficiency.

The fuel additive is used by adding the fuel additive to the fuel in an amount sufficient to increase fuel efficiency or to reduce pollutants. The terms enhanced and enhanced combustion refer to either of these effects. An example of reduced pollutants is a reduction of NOx and CO in an exhaust gas produced from an internal combustion engine. Advantageously, both of these effects are observed though the addition of the fuel additive of the current invention. A preferred embodiment includes the addition of the fuel additive to the fuel to achieve between about 50 and 150 ppm adenosine phosphate into the fuel though the addition of the fuel additive. Increased amounts of adenosine phosphate are effective as well. It is notable that a very cost-effective solution can be prepared with low weight percent of adenosine phosphate. Thus, a preferred embodiment includes adding the fuel additive to the fuel to achieve between around 1 ppm adenosine phosphate and 150 ppm adenosine phosphate in the hydrocarbon fuel.

Included in the invention is a process for enhancing fuel performance of a hydrocarbon fuel in a combustion system including the steps of providing the fuel additive described above in an amount effective to enhance fuel performance to the hydrocarbon fuel and combusting the hydrocarbon fuel with the fuel additive. The combustion system can be any means known to those with ordinary skill in the art for combusting hydrocarbon. The combustion system can include any of various internal combustion engines. In a preferred embodiment, this process is used with a liquid or liquefied hydrocarbon fuel. The result of adding the fuel additive to the hydrocarbon fuel is an enhanced fuel that has a substantial amount of hydrocarbon fuel suitable for combustion, and an amount of the fuel additive operable to enhance combustion. The enhanced fuel contains adenosine phosphate in an amount operable to reduce emissions or improve efficiency upon combustion of the enhanced fuel as compared to the combustion of the hydrocarbon fuel without the fuel additive. More preferably, the enhanced fuel contains adenosine phosphate of between about 1 and 150 ppm by weight of fuel.

An alternate embodiment of the invention includes a process for creating the enhanced fuel for use in a combustion system including the steps of adding the fuel additive to the hydrocarbon fuel in an amount effective to enhance fuel performance.

In the case of non-fuel soluble adenosine phosphate salts, a preferred embodiment of the process for creating the enhanced fuel includes dissolving or dispersing the adenosine phosphate salts in solvent or dispersion fluid to create an intermediate aqueous solution. In a preferred embodiment, dispersants can be added to the intermediate aqueous solution. Also, surfactants can be added to the intermediate aqueous solution with or without the dispersants.

Water contained in the solved or dispersion fluid is then removed in part or in full by means known in the art. Thermal means for water removal are particularly preferred. Removal of essentially all water is particularly preferred. The resulting dehydrated solution is the fuel additive to be added to the fuel to create the enhanced fuel.

In an additional embodiment, dispersants can be added to the enhanced fuel to maintain the adenosine phosphate or the fuel additive in appropriate dispersion within the fuel. Exemplary fuels include kerosene, diesel fuel, gasoline and residual fuels.

Examples of adenosine phosphates useful in the invention include ATP as well as adenosine diphosphate (ADP), adenosine monophosphate (AMP) and the salts of each. Adenosine phosphates can combine with other molecules to create useful compounds that are encompassed within the scope of the invention. Alkyl adenosine phosphates are also useful. Other adenosine phosphates are useful in this invention as well. Examples of preferred carrier fluids include water, liquid hydrocarbon, methyl alcohol, ethyl alcohol, propyl alcohol and other alcohols, with c1-c8 alcohols being preferred, glycols, ethyl glycol, propylene glycol, glycol ether EB, other glycol ethers, polyethers. Examples of preferred dispersion fluids include hydrocarbon fluids, other fluids in which the adenosine phosphate is not soluble but is dispersible, higher alkyl polyethers such polybutylene glycols, alkyl phenol polyethers, biodiesel, ethanol, and hydrocarbon blends. Examples of preferred dispersants include alkyl succinimide, branched alkyl benzene sulfonates, linear alkyl benzene sulfonates, polyalkyl succinanhydrides (PIBSA), and PIPSA-PAM.

### EXAMPLE 1

### Preparation of Adenosine Triphosphate Carrier Solution

A 12% aqueous solution of adenosine triphosphate disodium (ATP-2Na) can be prepared by addition of 120 grams of ATP-2Na (on dry basis) to 880 grams of water. The mixture is warmed to 35-40 deg C. to effect solution. The resulting solution then has a P content of about 19,000 ppm. Similarly, other salts of ATP can be used including other alkaline earth metals and ammonium salts.

### EXAMPLE 2

### Preparation of Adenosine Triphosphate Dispersion Fluid

An 800 ml beaker can be charged with 290.6 grams Star 4 base oil, 26.5 grams ATP carrier solution from Example 1 above, 54.6 grams of PIBSA-PAM dispersant solution (2.2% N) and 48.3 grams of kerosene. The mixture can then sheared to form an emulsion with Silverson LR 4TA high shear lab mixer. When a suitable emulsion had formed, the mixture can be heated over a period of time, while maintaining the high shear mixing, to remove the water. Heating is continued until the water content is at least lower than about 5,000 ppm. The resulting ATP particle dispersion is useful as a fuel treatment for internal combustion engines to reduce emissions and improve fuel economy. These dispersions can be used for the treatment of fuels for open flames and burners, including turbine engines. The fuel can be any fuel capable of sustaining combustion and can be either liquid, liquefied solid or gaseous.

### EXAMPLE 3

### Treatment of No 2. Diesel Fuel

Low sulfur No 2 diesel fuel can be treated with 1,000 ppm of the ATP dispersion fluid prepared in Example 2 above resulting in an ATP concentration in the fuel of about 100 ppm. The treated fuel is suitable to fuel a CAT D399 diesel engine gen-set rated at 1,100 hp and 800 kW. When operated under controlled load conditions, the fuel treatment can be expected to result in a reduction in fuel consumption, when compared to the gen-set operated with untreated fuel, of 6-7% and a reduction in the emissions of CO, NOx, and UBHC (unburned hydrocarbons) of 57%, 13% and 82% respectively. Opacity can be expected to improve by 43% and the exhaust gas temperature reduced by 26%.

While the invention has been shown or described in only some of its forms, it should be apparent to those skilled in the art that it is not so limited, but is susceptible to various changes without departing from the scope of the invention.

We claim:

1. A fuel additive for use with a hydrocarbon fuel, the fuel additive comprising an amount of adenosine phosphate, the amount of fuel additive being operable to enhance combustions when added to the hydrocarbon fuel to create an enhanced hydrocarbon fuel in comparison to combustion of the hydrocarbon fuel without the fuel additive.

2. The fuel additive of claim 1 wherein the fuel additive includes the adenosine phosphate in solution with a carrier fluid to create adenosine phosphate solution.

3. The fuel additive of claim 2 wherein the carrier fluid is aqueous.

4. The fuel additive of claim 2 wherein the carrier fluid is a hydrocarbon fluid.

5. The fuel additive of claim 4 wherein the hydrocarbon fluid is an amount of the hydrocarbon fuel.

6. The fuel additive of claim 2 further comprising a dispersion fluid added to the adenosine phosphate solution to create a treated adenosine phosphate solution, the dispersion fluid being operable to maintain the adenosine phosphate within the dispersion fluid in at least a partially dispersed state and the dispersion fluid being at least partially miscible in the hydrocarbon fuel.

7. The fuel additive of claim 1 wherein the adenosine phosphate is in the form of a cationic salt.

8. The fuel additive of claim 7 wherein the adenosine phosphate includes a cation selected from the alkali metals or Group IA elements.

9. The fuel additive of claim 7 wherein the adenosine phosphate includes an ammonium cation.

10. The fuel additive of claim 1 further comprising a dispersant agent such that the adenosine phosphate is operable of being maintained in at least a partially dispersed state in the carrier fluid.

11. The fuel additive of claim 1 wherein the adenosine phosphate is present in the fuel additive in a range of between approximately 1 ppm and 150 ppm adenosine phosphate to hydrocarbon fuel.

12. A process for forming a fuel additive for use in a hydrocarbon fuel, the process comprising the steps adding an amount of adenosine phosphate into the hydrocarbon fuel, the amount of adenosine phosphate being operable to enhance combustions when added to the hydrocarbon fuel to create an enhanced hydrocarbon fuel in comparison to combustion of the hydrocarbon fuel without the fuel additive.

13. The process of claim 12 further comprising the step of adding the adenosine phosphate to a carrier fluid to form an adenosine phosphate solution prior to mixing with the hydrocarbon fuel.

14. The process of claim 13 wherein the carrier fluid is aqueous.

15. The process of claim 13 wherein the carrier fluid is a hydrocarbon fluid.

16. The process of claim 13 further comprising the step of adding a dispersion fluid to the adenosine phosphate solution to create a treated adenosine phosphate solution, the dispersion fluid being operable to maintain the adenosine phosphate within the dispersion fluid in at least a partially dispersed state and the dispersion fluid being at least partially miscible in the hydrocarbon fuel.

17. The process of claim 16 further comprising the step of at least partially dehydrating the treated adenosine phosphate solution to remove a substantial portion of water present in the treated adenosine phosphate solution prior to addition of the adenosine phosphate in the form of the treated adenosine phosphate solution into the hydrocarbon fuel.

18. The process of claim 16 further comprising the step of heating the treated adenosine phosphate solution to remove a substantial portion of volatile components present in the treated adenosine phosphate solution prior to addition of the adenosine phosphate in the form of the treated adenosine phosphate solution into the hydrocarbon fuel.
